(19) United States
(12) Patent Application Publication
(10) Pub. No.: US 2024/0008716 A1
(43) Pub. Date: Jan. 11, 2024

(54) DISTAL END PORTION OF ENDOSCOPE AND ENDOSCOPE

(71) Applicant: OLYMPUS CORPORATION, Tokyo (JP)

(72) Inventor: Tadahiro NAKANO, Tokyo (JP)

(73) Assignee: OLYMPUS CORPORATION, Tokyo (JP)

(21) Appl. No.: 18/371,613

(22) Filed: Sep. 22, 2023

Related U.S. Application Data
(63) Continuation of application No. PCT/JP2021/023844, filed on Jun. 23, 2021.

(51) Int. Cl. A61B 1/00; A61B 1/018

(52) U.S. Cl. CPC A61B 1/00098; A61B 1/00124; A61B 1/0011; A61B 1/00137; A61B 1/018

(57) ABSTRACT

A distal end portion of an endoscope includes: a moving mechanism configured to change a protruding direction of a treatment instrument; a base made of metal and including a supporting portion configured to rotationally support the moving mechanism; a cover member made of resin, the cover member being fitted to the base so as to cover the moving mechanism and including an opening portion formed at a position at which the moving mechanism is placed; an electronic component disposed in the cover member; and a wiring pattern electrically connected to the electronic component, the wiring pattern being provided in the cover member.

### CROSS-REFERENCE TO RELATED APPLICATION

This application is a continuation application of PCT/JP2021/023844 filed on Jun. 23, 2021, the entire contents of which are incorporated herein by this reference.

### BACKGROUND OF THE INVENTION

### 1. Field of the Invention

The present invention relates to a distal end portion of an endoscope and an endoscope. The distal end portion of the endoscope includes, at a distal end part thereof, a moving mechanism configured to change a direction of a treatment instrument or the like.

### 2. Description of the Related Art

In recent years, side-view endoscopes, which are medical devices, have been known. Such side-view endoscopes include an objective lens, an illumination lens, a treatment instrument lead-out port, and the like on a side surface of a distal end of a long insertion portion as an insertion body. Side-view endoscopes are used for observation in a non-parallel direction with respect to an insertion direction of an insertion portion inside a subject.

For example, Japanese Patent Application Laid-Open Publication No. 2019-115562 discloses an endoscope configured such that a raising base that changes a protruding direction of a treatment instrument at a distal end portion of the endoscope is detached after an endoscopic examination, to thereby improve a cleaning property of the endoscope.

### SUMMARY OF THE INVENTION

A distal end portion of an endoscope according to one aspect of the present invention includes: a moving mechanism configured to change a protruding direction of a treatment instrument; a base made of metal and including a supporting portion configured to rotationally support the moving mechanism; a cover member made of resin, the cover member being fitted to the base so as to cover the moving mechanism and including an opening portion formed at a position at which the moving mechanism is placed; an electronic component disposed in the cover member; and a wiring pattern electrically connected to the electronic component, the wiring pattern being provided in the cover member.

An endoscope according to one aspect of the present invention includes: an insertion body configured to be inserted into a subject; a moving mechanism configured to change a protruding direction of a treatment instrument configured to be inserted through the insertion body; a base made of metal and including a supporting portion configured to rotationally support the moving mechanism; a cover member made of resin, the cover member being fitted to the base so as to cover the moving mechanism and including an opening portion formed at a position at which the moving mechanism is placed; an electronic component disposed in the cover member; and a wiring pattern electrically connected to the electronic component, the wiring pattern being provided in the cover member.

### BRIEF DESCRIPTION OF THE DRAWINGS

FIG. 1 is an external plan view of an endoscope according to a first embodiment of the present invention.

FIG. 2 is a perspective view showing a configuration of a distal end part of an insertion portion as an insertion body according to the first embodiment of the present invention.

FIG. 3 is a plan view showing the configuration of the distal end part of the insertion portion as the insertion body according to the first embodiment of the present invention.

FIG. 4 is a perspective view showing a configuration of a distal end cover according to the first embodiment of the present invention.

FIG. 5 is a rear view showing the configuration of the distal end cover according to the first embodiment of the present invention.

FIG. 6 is a top view showing the configuration of the distal end cover according to the first embodiment of the present invention.

FIG. 7 is a plan view partially showing a configuration of an electric circuit formed in the distal end cover according to the first embodiment of the present invention.

FIG. 8 is a perspective view partially showing the configuration of the electric circuit formed in the distal end cover according to the first embodiment of the present invention.

FIG. 9 is an exploded perspective view partially showing an illumination optical system, an image pickup optical system, and the distal end cover according to the first embodiment of the present invention.

FIG. 10 is a perspective view partially showing a state where the illumination optical system and the image pickup optical system are mounted in the distal end cover according to the first embodiment of the present invention.

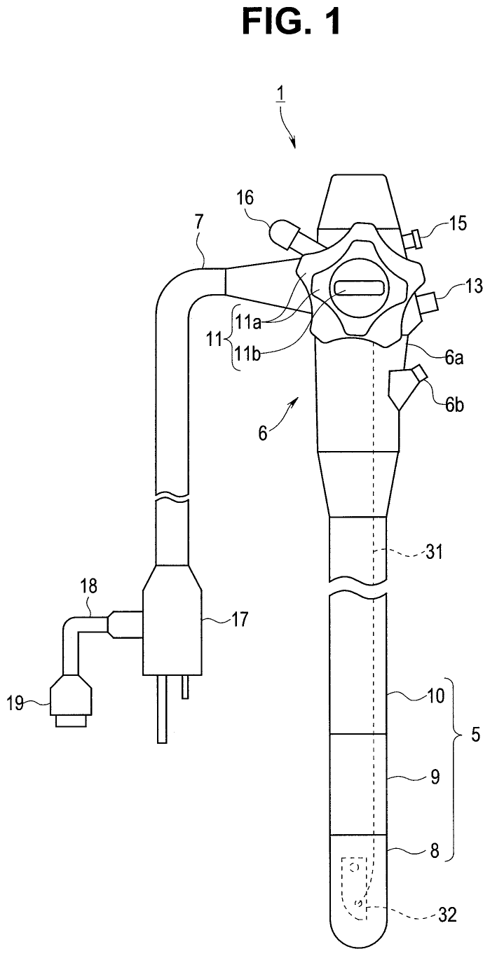

FIG. 1I is a perspective view from the right side, which shows a configuration of a distal-most bending piece according to the first embodiment of the present invention.

FIG. 12 is a perspective view from the left side, which shows the configuration of the distal-most bending piece according to the first embodiment of the present invention.

FIG. 13 is a plan view showing a state before the distal end cover and the distal-most bending piece are fitted to each other in the first embodiment of the present invention.

FIG. 14 is a perspective view partially showing the state before the distal end cover and the distal-most bending piece are fitted to each other in the first embodiment of the present invention.

FIG. 15 is a partial perspective view showing a connection of cables in the first embodiment of the present invention.

FIG. 16 is a perspective view partially showing a state where the distal end cover and the distal-most bending piece are fitted to each other in the first embodiment of the present invention.

FIG. 17 is a perspective view partially showing a state where a filling agent is applied around the illumination optical system and the image pickup optical system in the first embodiment of the present invention.

FIG. 18 is a top view showing the state where the distal end cover and the distal-most bending piece are fitted to each other in the first embodiment of the present invention.

FIG. 19 is a top view showing a configuration of a distal end cover according to a second embodiment of the present invention.

FIG. 20 is bottom view showing the configuration of the distal end cover according to the second embodiment of the present invention.

FIG. 21 is a perspective view showing a configuration of a distal end cover according to the second embodiment of the present invention.

FIG. 22 is a cross-sectional view showing the configuration of the distal end cover according to the second embodiment of the present invention.

FIG. 23 is a plan view partially showing a configuration of an electric circuit formed in the distal end cover according to the second embodiment of the present invention.

FIG. 24 is a cross-sectional view partially showing a side of a solder bump of an illumination optical system or an image pickup optical system, as an electronic component which is formed by insert molding in the distal end cover according to the second embodiment of the present invention.

FIG. 25 is a cross-sectional view partially showing a state where a hole portion is formed in the distal end cover by laser light being applied to the solder bump of the illumination optical system or the image pickup optical system in the second embodiment of the present invention.

FIG. 26 is a cross-sectional view partially showing a state where a wiring pattern, which is electrically connected to the solder bump of the illumination optical system or the image pickup optical system, is formed in the distal end cover according to the second embodiment of the present invention.

FIG. 27 is a top view showing a configuration of an optical unit in a first modification example.

FIG. 28 is a cross-sectional view of the configuration of the optical unit, which is taken along the line XXVIII-XXVIII in FIG. 27, in the first modification example.

FIG. 29 is an exploded perspective view partially showing the optical unit and a distal end cover in the first modification example.

FIG. 30 is a perspective view partially showing a state where an illumination optical system and the image pickup optical system of the optical unit are mounted in the distal end cover and a filling agent is applied around the illumination optical system and the image pickup optical system, in the first modification example.

FIG. 31 is a top view showing a distal end cover constituted of two resin members, in a second modification example.

FIG. 32 is a rear view showing the distal end cover constituted of the two resin members, in the second modification example.

FIG. 33 is a cross-sectional view showing a configuration of a distal end cover in which an illumination optical system formed by insert molding is a light guide bundle, in a third modification example.

FIG. 34 is a cross-sectional view showing the configuration of the distal end cover including an illumination optical system placing hole in which the light guide bundle is arranged, in the third modification example.

### DETAILED DESCRIPTION OF THE PREFERRED EMBODIMENT(S)

Hereinafter, a distal end portion of an endoscope and an endoscope according to one aspect of the present invention will be described with reference to drawings. Note that, in the description below, each of the drawings based on each embodiment is a pattern diagram, and care should be taken to the fact that the relationship between the thicknesses and widths of the respective parts, a ratio of the thickness of a certain part to that of another part, and the like are different from the actual ones, and there is a case where the respective drawings include parts in which the relationships and ratios among the dimensions are different.

In addition, the endoscope, which is a medical device, in the configuration description below will be described by taking, what is called, a flexible endoscope as an example. Such a flexible endoscope has a flexible insertion portion configured to be inserted into the bronchus, the urinary organ, the part from the esophagus to the stomach, the small intestine, the large intestine, and the like, in the living body. Note that the endoscope as a medical device is also applicable to, what is called, a rigid endoscope having a rigid insertion portion to be used for surgery. In addition, in the description below, the technology of the present invention is applicable also to an industrial endoscope.

### First Embodiment

As shown in FIG. 1, an endoscope 1 according to the present embodiment includes an insertion portion 5 which is an insertion body to be inserted into a subject, an operation portion 6, and a universal cable 7. The insertion portion 5 is an elongated member configured to be inserted into a site to be observed from the distal end side in the longitudinal axis direction. The insertion portion 5 includes, in a linked manner, a distal end portion 8, a bending portion 9, and a flexible tube portion 10.

The distal end portion 8 incorporates inside thereof an illumination optical system including a light guide, and an image pickup optical system including an image pickup apparatus (neither of them is shown here). The distal end portion 8 includes an observation window of the image pickup optical system and an illumination window of the illumination optical system that have a predetermined angle with respect to an insertion direction of the insertion portion 5. In addition, the distal end portion 8 includes a raising base (forceps elevator) 32 which is a moving portion as a direction changing section configured to raise a treatment instrument to change a protruding direction of the treatment instrument in an observation direction. In other words, the present embodiment relates to a technology applied to a side-view endoscope or an oblique-view endoscope.

The raising base 32 is connected to a raising base operation wire (hereinafter, shortly referred to as a wire) 31. The wire 31 is a pulling and relaxing member as a long member that is inserted through the insertion portion 5 and the operation portion 6. The wire 31 is pulled and relaxed, to thereby cause the raising base 32 to be raised and lowered. Note that the wire 31 is pulled and relaxed by operating a raising base operation lever 16.

The bending portion 9 is configured to be bendable in four directions, i.e., up, down, left, and right directions, for example. The flexible tube portion 10 is a long and flexible tubular member.

The operation portion 6 includes a grasping portion Ca. The grasping portion 6a is connected to the proximal end portion of the insertion portion 5. A treatment instrument insertion port 6b is disposed on the grasping portion Ca. The operation portion 6 is provided with a bending operation portion 11, a gas/liquid feeding button 13, a cleaning tube attaching pipe sleeve 15, and the like. In addition, the operation portion 6 is provided with the raising base operation lever 16 as an operation member.

The bending operation portion 11 includes a bending operation knob 11a for performing bending operation of the bending portion 9 of the insertion portion 5 and a fixing lever 11b for fixing the bending operation knob 11a at a desired rotation position.

The universal cable 7 is extended from a side surface of the operation portion 6. The universal cable 7 includes, at an end portion thereof, an endoscope connector 17 that is connected to a light source apparatus as an external apparatus. A signal transmission cable 18 is extended from a side portion of the endoscope connector 17. On the other end side of the signal transmission cable 18, an electrical connector 19 configured to be connected to a video processor is provided.

Here, a detailed description will be made on a structure of the distal end portion 8 of the insertion portion 5 as the insertion body of the present embodiment.

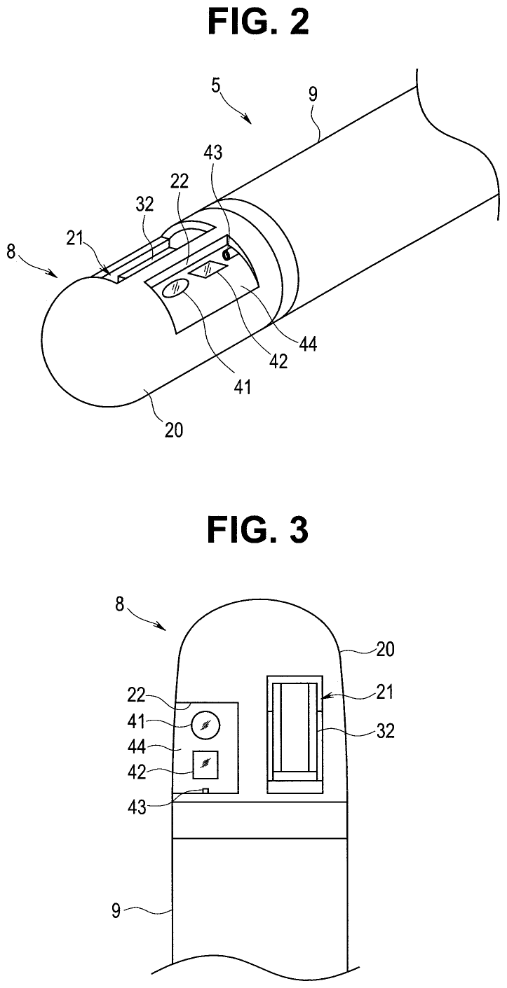

As shown in FIG. 2 and FIG. 3, the distal end portion 8 is disposed at the distal end of the insertion portion 5. The distal end portion 8 includes a distal end cover 20 which is a cover member made of synthetic resin. On the distal end cover 20, an illumination window 41 of the illumination optical system and an observation window 42 of the image pickup optical system are provided. The distal end cover 20 is fitted, to be adhered and fixed to a distal-most bending piece 50 (see FIG. 11 and FIG. 12) as a metal frame to be described later.

Note that the distal end cover 20 of the present embodiment is a rigid resin component configured of a molded interconnect device (MID).

The illumination window 41 and the observation window 42 are arranged side by side along an insertion axis of the insertion portion 5, which is a longitudinal direction of the distal end cover 20. An opening portion 43 of a gas/liquid feeding conduit is provided toward the illumination window 41 and the observation window 42, In addition, a filling agent 44 such as an underfill agent that does not have a light transmitting property is applied around the illumination window 41 and the observation window 42 in the distal end cover 20 such that respective surfaces of the illumination window 41 and the observation window 42 are exposed.

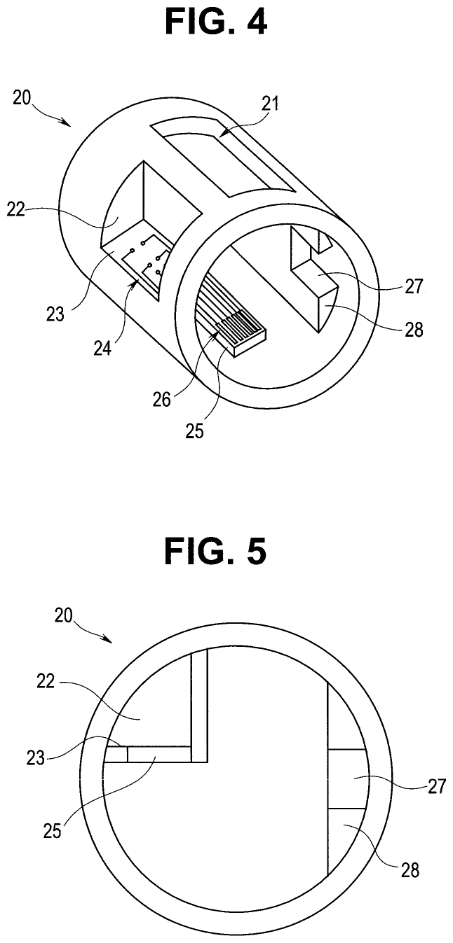

As shown in FIG. 4 and FIG. 5, the distal end cover 20 is a hollow resin member formed by injection molding, for example. The distal end cover 20 includes an opening portion 21 and a recessed portion 22. In the recessed portion 22, the illumination window 41 and the observation window 42 are arranged. In the opening portion 21, the raising base 32 is arranged so as to locate in the vicinity of the illumination window 41 and the observation window 42.

Note that, in the present embodiment, when the distal end cover 20 is arranged as shown in FIG. 3, the illumination window 41 and the observation window 42 are arranged on the left side when viewed from the direction in which the illumination window 41 and the observation window 42 are exposed. On the right side of the illumination window 41 and the observation window 42, the opening portion 21 of a raising base placing portion is arranged.

The distal end cover 20 includes, inside thereof, a wall portion 28 on which a fitting groove 27 is formed. The fitting groove 27 is configured to fit to raising base pivotably-supporting portions 53 which are supporting portions configured to rotationally support the raising base 32. The wall portion 28 is formed at substantially the center of an inner surface of a side portion on the right side when the distal end cover 20 is viewed from the rear surface side.

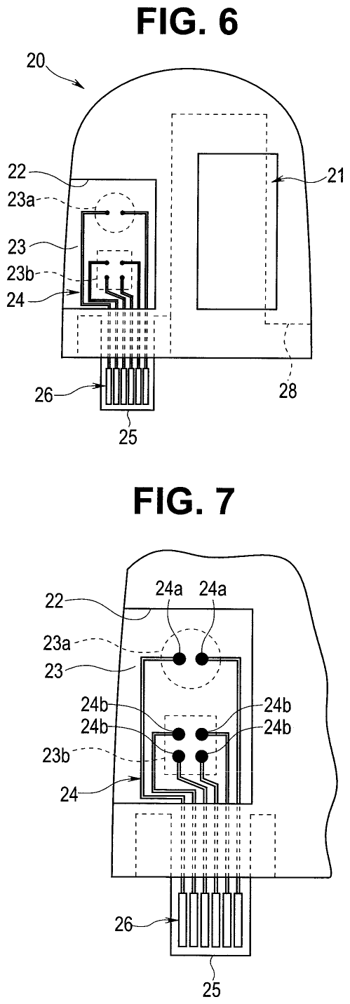

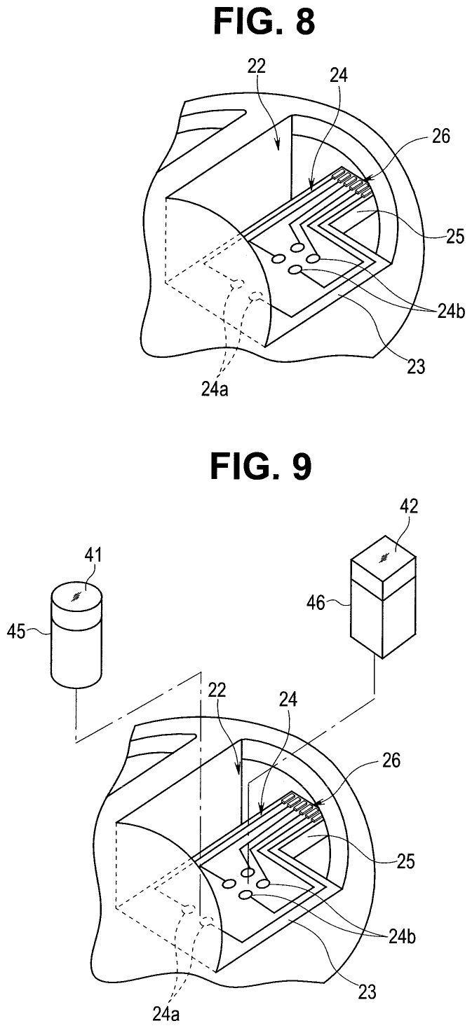

As shown in FIG. 6 to FIG. 8, the distal end cover 20 includes a plate-shaped cable connecting portion 25 extended backward from a bottom surface 23 of the recessed portion 22. A plurality of wiring patterns 24 as conductive patterns are provided on the bottom surface 23 of the recessed portion 22 and the cable connecting portion 25.

Note that the cable connecting portion 25 has a plane, as a top surface thereof, which is continuous with the bottom surface 23. The plurality of wiring patterns 24 are formed on the plane of the cable connecting portion 25. In addition, on the plane of the cable connecting portion 25, a plurality of cable connecting terminals 26 are arranged side by side. The plurality of cable connecting terminals 26 are electrically connected respectively to the plurality of wiring patterns 24.

The distal end cover 20 includes, on the bottom surface 23 of the recessed portion 22, an illumination optical system placing region 23a and an image pickup optical system placing region 23b. In the illumination optical system placing region 23a, an illumination optical system terminal 24a is formed at each of the end portions of the two wiring patterns 24. In addition, in the image pickup optical system placing region 23b, an image pickup optical system terminal 24b is formed at each of the end portions of the four wiring patterns 24.

In other words, the distal end cover 20 includes, on the bottom surface of the recessed portion 22, the two illumination optical system terminals 24a and the four image pickup optical system terminals 24b. The distal end cover 20, includes the plurality of wiring patterns 24, the number of which is six in total, and the plurality of cable connecting terminals 26, the number of which is six in total. The six wiring patterns 24 and the six cable connecting terminals 26 are electrically connected to the two illumination optical system terminals 24a and the four image pickup optical system terminals 24b.

Note that, in the distal end cover 20, the plurality of wiring patterns 24, the illumination optical system terminals 24a, the image pickup optical system terminals 24b, and the cable connecting terminals 26 are formed by using the MID technology for performing plating processing on a part activated by applying laser light. Thus, in the distal end cover 20, the electric circuits are formed on the bottom surface 23 of the recessed portion 22 and the surface of the cable connecting portion 25.

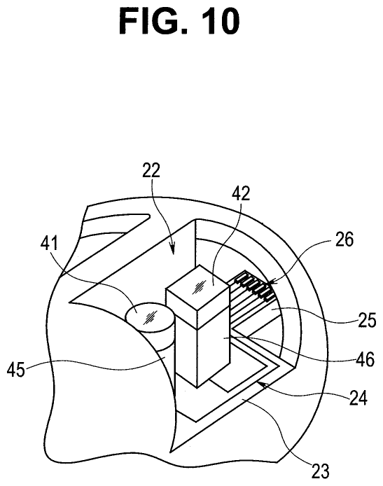

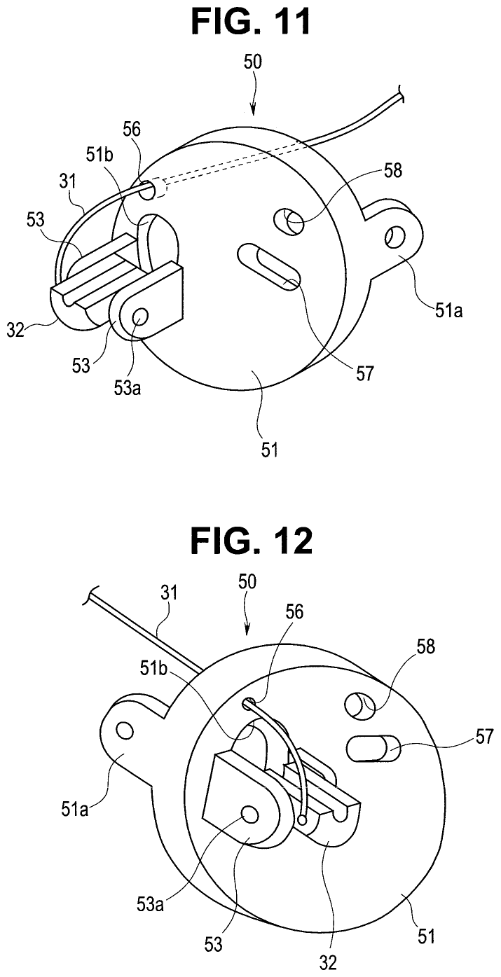

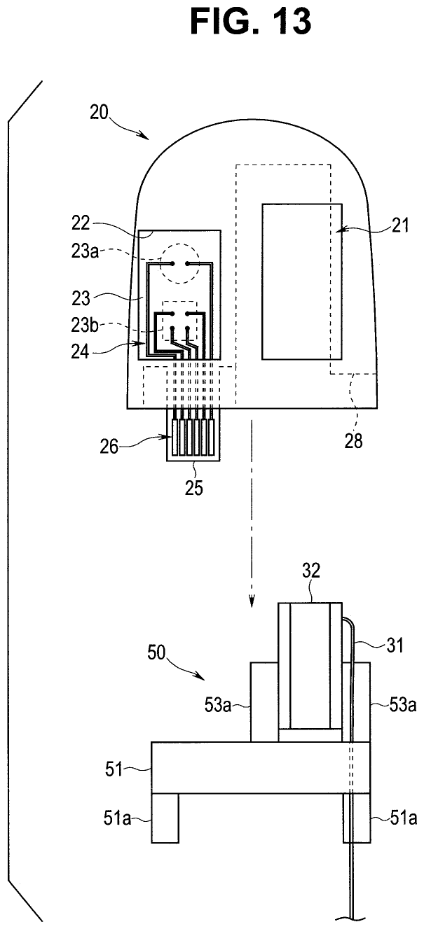

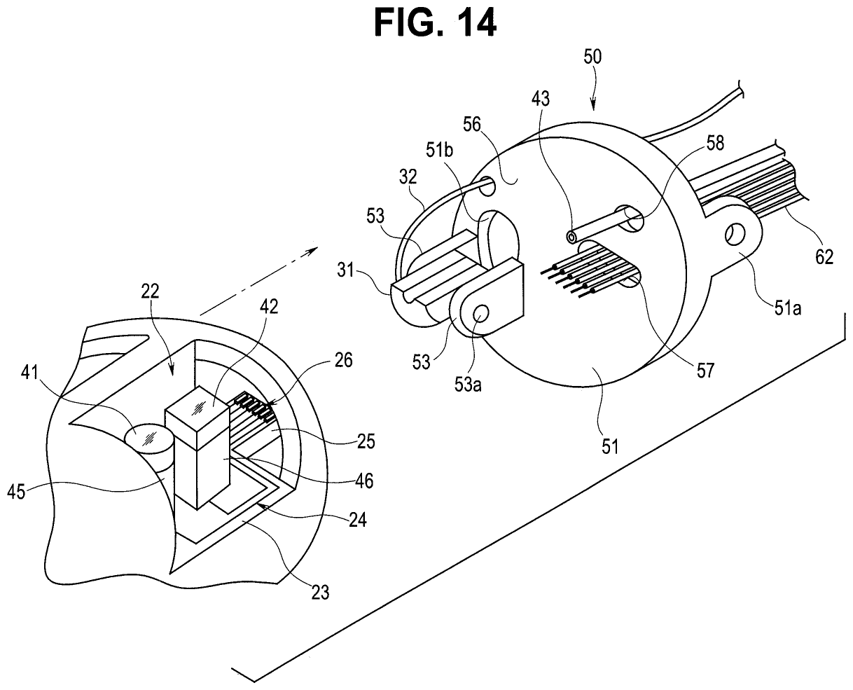

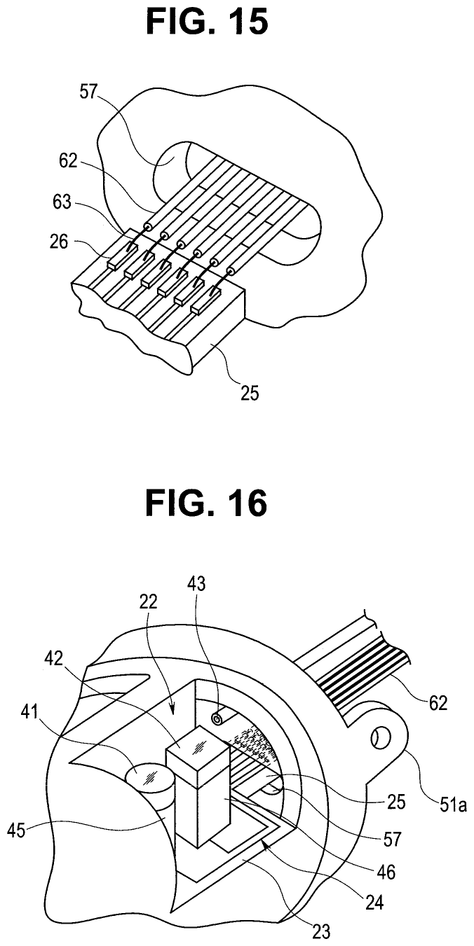

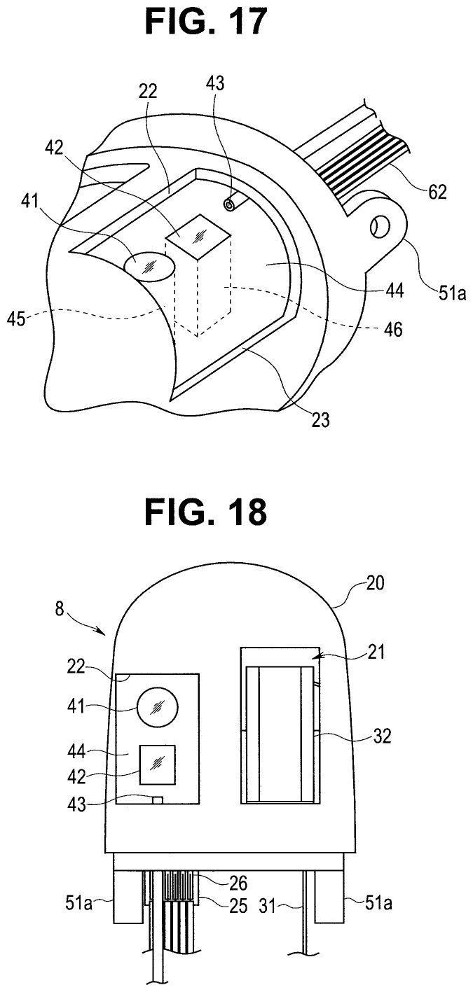

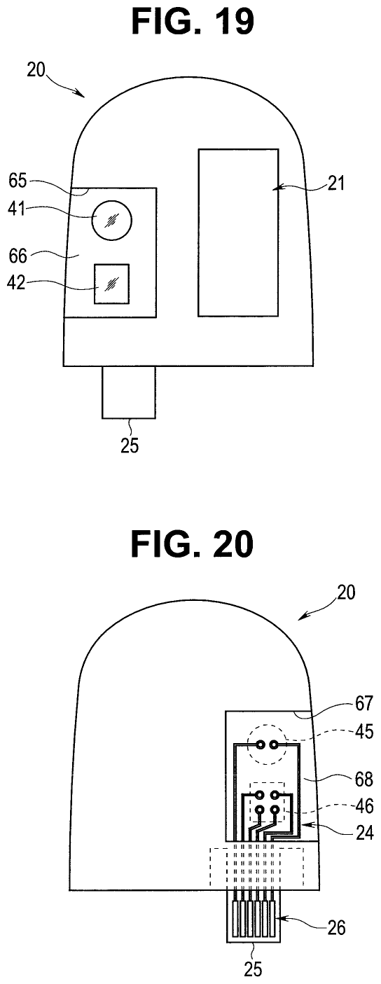

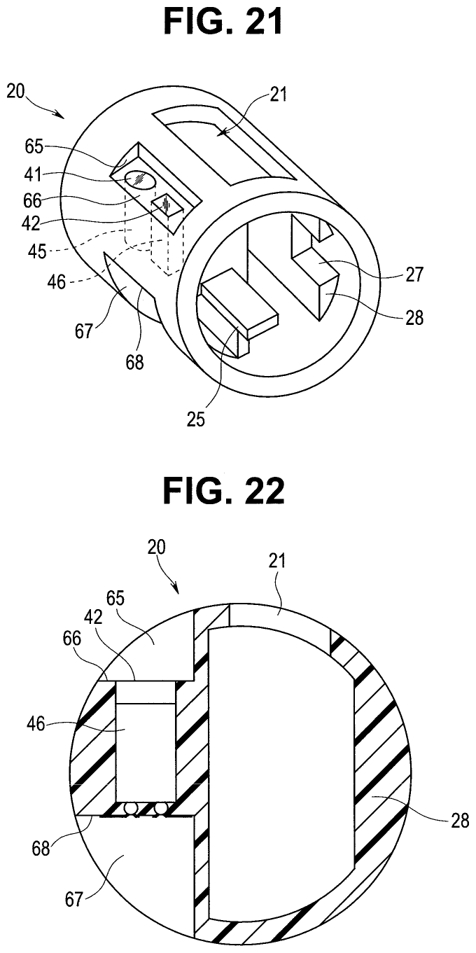

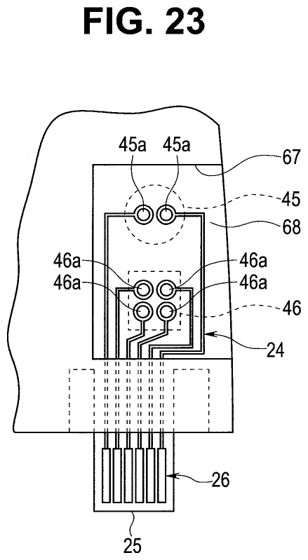

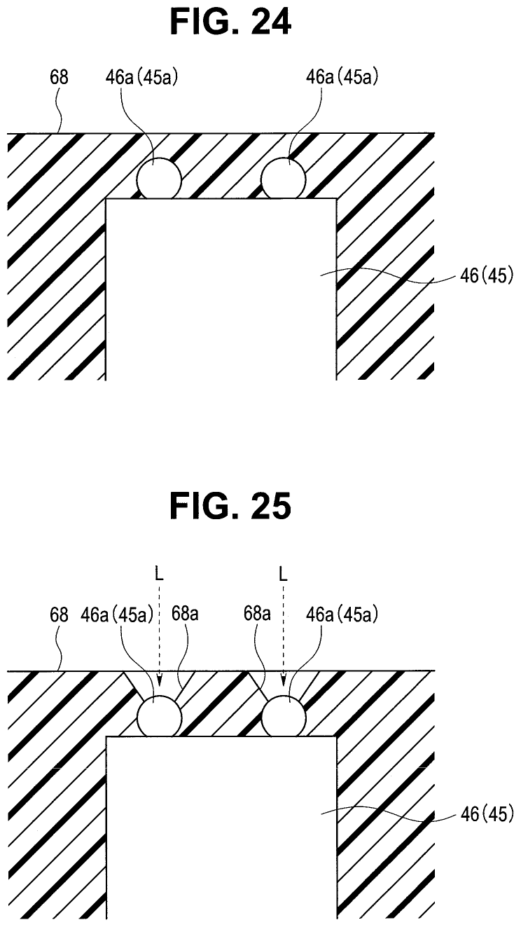

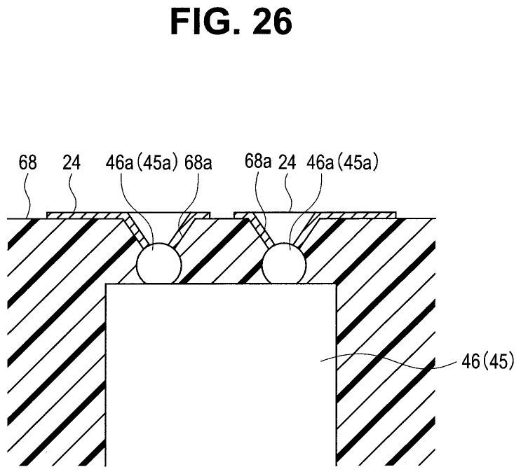

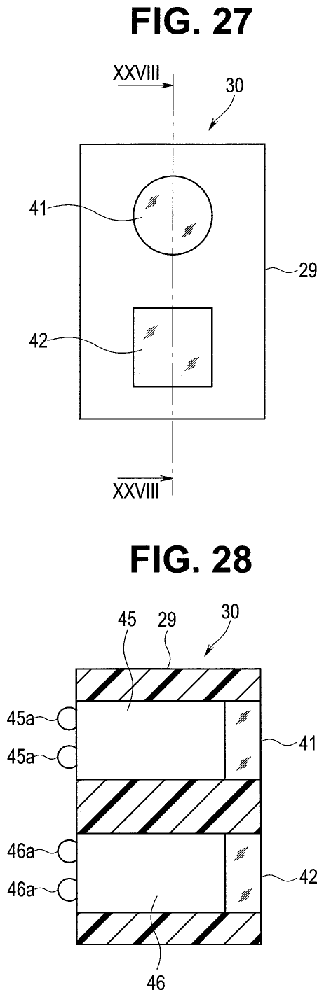

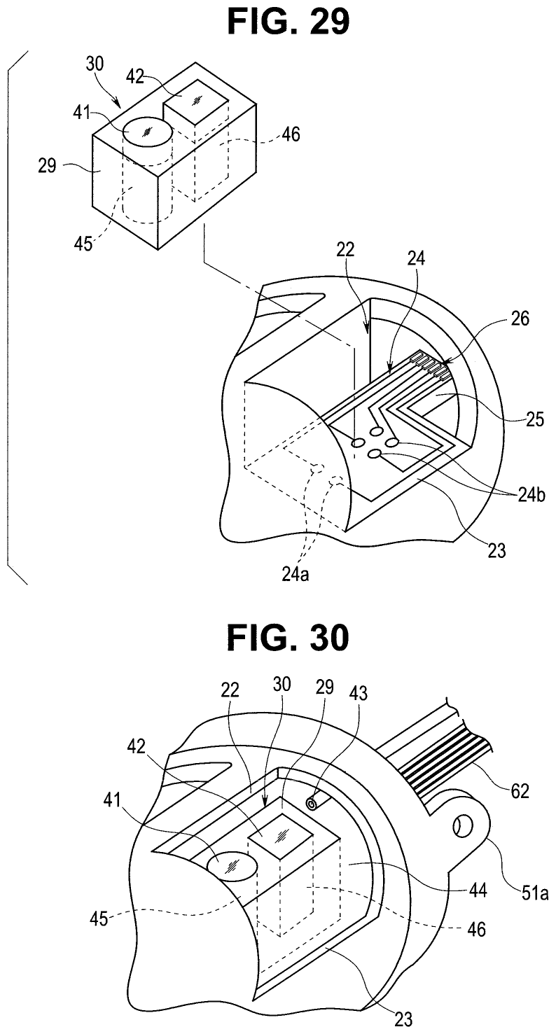

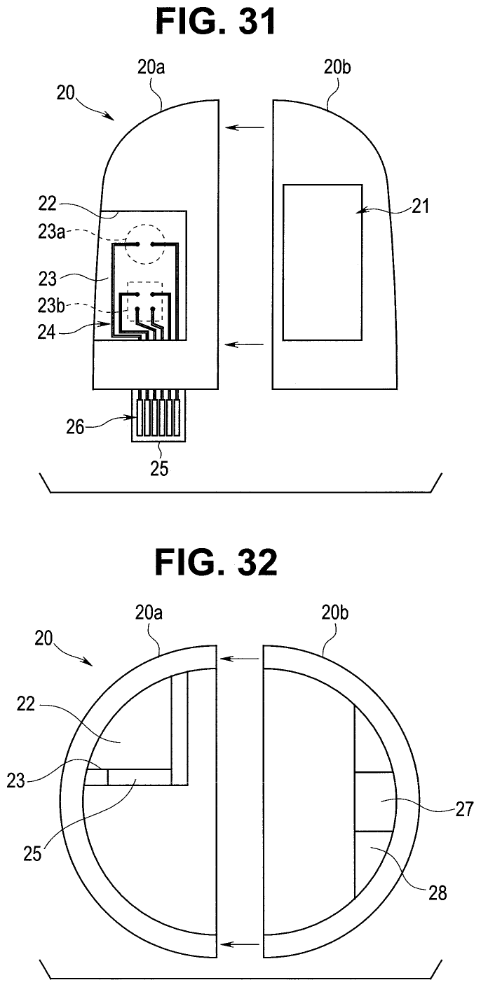

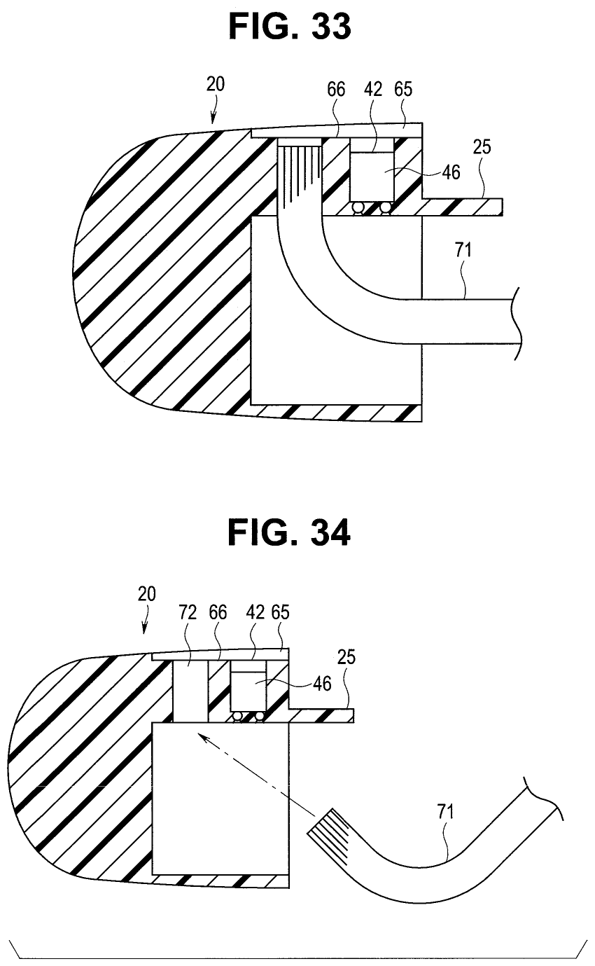

In the distal end cover 20 thus configured, an illumination module 45 and a camera module 46 are mounted on the bottom surface 23 of the recessed portion 22, as shown in FIG. 9 and FIG. 10. The illumination module 45 is an illumination optical system with which the illumination window 41 is integrally formed, and the camera module 46 is an image pickup unit with which the observation window 42 is integrally formed.

The illumination module 45 has two electrodes electrically connected, by solder or the like, respectively to the two illumination optical system terminals 24a in the illumination optical system placing region 23a. In addition, the camera module 46 has four electrodes electrically connected, by solder or the like, respectively to the four image pickup optical system terminals 24b in the image pickup optical system placing region 23b.

Note that the illumination module 45 is an LED module with which the illumination window 41 as a cover glass is integrally formed. The camera module 46 is formed by an image pickup lens unit, the observation window 42, and an image pickup device being integrally stacked. For example, the image pickup lens unit is constituted of a lens stack fabricated using a wafer level optics technology. The observation window 42 configures a cover glass of the camera module 46. The image pickup device is a CCD or a CMOS, and is provided with a lens unit pasted on the observation window 42 through an adhesion layer. Thus, the camera module 46 is configured by a CSP (chip size package).

Such a camera module 46 is manufactured, for example, by fabricating a plurality of lens wafers each including lenses formed on a base material such as a glass substrate, stacking these lens waters, and dicing the lens wafers. Thus, the camera module 46 has a rectangular shape in a planar view, and the entirety thereof is formed in a very small substantially rectangular parallelepiped shape, one side of which is several millimeters, without a lens frame.

As shown in FIG. 11 and FIG. 12, the distal-most bending piece 50 to be fitted to the distal end cover 20 is a metal body formed in substantially a disk shape. The distal-most bending piece 50 includes a base 51 as a main body and two piece-connecting portions 51a extended backward respectively from both side portions of the base 51,

The base 51 includes the two raising base pivotably-supporting portions 53 formed so as to protrude forward from the distal end surface. The raising base 32 made of metal, which is pivotably supported by a pivot shaft 53a, is provided between the two raising base pivotably-supporting portions 53. Note that, the base 51 includes an opening 51b formed between the two raising base pivotably-supporting portions 53. The opening Sib serves as a treatment instrument insertion port.

The pulling wire 31 for raising and lowering the raising base 32 is connected to the raising base 32. The pulling wire 31 is inserted through a wire insertion hole 56 formed in the base 51, and extended backward. In addition, the base 51 includes a cable connecting portion insertion hole 57 having a long hole shape and a liquid feeding conduit insertion hole 58.

The structure of the distal end portion of the insertion portion 5, which is configured as described above by the distal end cover 20 and the distal-most bending piece 50, is formed by the distal end cover 20 being fitted and mounted to the distal-most bending piece 50 so as to cover the raising base 32, as shown in FIG. 13.

At this time, in the distal-most bending piece 50, six cables 62 are inserted through the cable connecting portion insertion hole 57, as shown in FIG. 14. In addition, in the distal-most bending piece 50, a liquid feeding conduit 61 is inserted through the liquid feeding conduit insertion hole 58. Next, as shown in FIG. 15, core wires 63 of the cables 62 are connected, by solder or the like, respectively to the six cable connecting terminals 26 formed at the cable connecting portion 25 of the distal end cover 20.

Then, the distal end cover 20 and the distal-most bending piece 50 are fitted to each other and secured by an adhesive or the like. At this time, the cable connecting portion 25 of the distal end cover 20 is inserted into the cable connecting portion insertion hole 57 of the distal-most bending piece 50. Note that, although not shown, the fitting groove 27 of the distal end cover 20 is fitted to the raising base pivotably-supporting portion 53 on the outer side of the distal-most bending piece 50 (see FIG. 4).

The distal end cover 20 is held such that the distal end opening of the liquid feeding conduit 61 faces the illumination window 41 of the module 45 and the observation window 42 of the camera module 46. Then, as shown in FIG. 17, in the distal end cover 20, the filling agent 44 as a filler like an underfill agent is applied to the recessed portion 22 to be molded, and solidified by heat processing or the like. Thus, the distal end portion 8 is configured by the distal end cover 20 and the distal-most bending piece 50 being assembled as shown in FIG. 18.

In the distal end portion 8 of the insertion portion 5 of the endoscope 1 configured as described above, the raising base 32, which is a moving mechanism for raising and lowering the treatment instrument and which requires a mechanical strength, and the distal-most bending piece 50 in which the raising base pivotably-supporting portions 53 that rotationally support the raising base 32 and the base 51 are integrally formed are made of metal, and the distal end cover 20 which is a cover member that covers the raising base 32 is made of resin. With such a configuration, the distal end portion 8 ensures both the strength and the durability.

Then, in the distal end cover 20 of the distal end portion 8, the plurality of wiring patterns 24 and the plurality of cable connecting terminals 26 are formed in the recessed portion 22 and on the surface of the cable connecting portion 25 by using the MIT) technology. The layout in the distal end cover 20 of the distal end portion 8 is such that the illumination module 45 and the camera module 46, which are electronic components electrically connected to the wiring patterns 24, are mounted in the recessed portion 22.

As a result, the endoscope according to the present embodiment is capable of suppressing troubles of processing the distal end portion 8 of the insertion portion 5 as the insertion body and assembling various kinds of components, to thereby be capable of reducing the manufacturing cost.

Thus, in the endoscope 1, the distal end portion 8 of the insertion portion 5 includes, at the metal portion, the moving mechanism such as the raising base 32 configured to change the direction of the treatment instrument, and includes the distal end cover 20 in which the illumination module 45 as the illumination optical system and the camera module 46 as the image pickup optical system are mounted in the vicinity of the moving mechanism. With such a configuration, the endoscope 1 is capable of achieving the ensuring of the mechanical strength and the durability simultaneously with the reduction of the manufacturing cost.

Thus, the endoscope 1 is a side-view or an oblique-view endoscope configured mostly as a duodenum scope in which a field-of-view direction has an angle with respect to the insertion direction, and contributes to single use of the endoscope by ensuring the mechanical strength and the durability and reducing the manufacturing cost. Note that, the moving mechanism configured to change the protruding direction of the treatment instrument from the distal end portion of the endoscope 1 can be applied also to various kinds of medical devices such as a surgical manipulator, and various kinds of industrial devices to be used for repair in an industrial use. The instrument whose protruding direction from the distal end portion of the endoscope 1 is changed is not limited to the treatment instrument, and includes another endoscope, an optical probe, and the like.

### Second Embodiment

Next, a configuration in the second embodiment will be described. Note that, in the description of the present embodiment, the same constituent elements as those in the above-described first embodiment are attached with the same reference signs and detailed descriptions thereof will be omitted.

An endoscope 1 of the present embodiment is different from the one in the first embodiment in a configuration of a distal end cover 20 provided to a distal end portion 8 of an insertion portion 5 as an insertion body. The endoscope 1 of the present embodiment includes the distal end cover 20 formed integrally with an illumination module 45 as an illumination optical system and a camera module 46 as an image pickup optical system, by using an insert molding technology in which, at the time of resin molding, the illumination module 45 and the camera module 46 are put into a mold for the resin molding, not shown, and thereafter a resin material is poured into the mold.

Specifically, as shown in FIGS. 19 to 22, the distal end cover 20 includes, on the upper surface thereof, a bottom surface 66 of a recessed portion 65. On the bottom surface 66, an illumination window 41 and an observation window 42 are exposed (see FIG. 19, FIG. 21, and FIG. 22).

In addition, the distal end cover 20 includes, also on the lower surface thereof, a recessed portion 67 (see FIG. 20 to FIG. 22) at a position corresponding to the position where the illumination module 45 and the camera module 46 are put by insert molding. A plurality of wiring patterns 24 are provided on a bottom surface 68 of the recessed portion 67 which is on the lower surface side and on a plate-shaped cable connecting portion 25 extended backward from the bottom surface 68.

Note that the cable connecting portion 25 has a plane, which serves as the rear surface (lower surface) thereof, continuous with the bottom surface 68. On the plane, the plurality of wiring patterns 24 are formed. In addition, on the plane of the cable connecting portion 25, a plurality of cable connecting terminals 26 are arranged side by side. The plurality of cable connecting terminals 26 are electrically connected respectively to the plurality of wiring patterns 24.

In addition, as shown in FIG. 23, the plurality of wiring patterns 24 are connected, on the bottom surface 68 of the recessed portion 67, electrically to two solder bumps 45a of the illumination module 45 and four solder bumps 46a of the camera module 46.

Note that the illumination module 45 and the camera module 46 are put in the distal end cover 20 by insert molding, as shown in FIG. 24, Therefore, as shown in FIG. 25, laser light L is applied from the side of the bottom surface 68 of the recessed portion 67 toward the respective solder bumps 45a and 46a, to remove the thickness of the bottom surface 68, to thereby form six hole portions 68a for allowing the solder bumps 45a and 46a to be respectively exposed.

Then, in the distal end cover 20, the electric circuits are formed by using the MID technology in which plating processing is performed on the parts activated by applying the laser light. In other words, in the distal end cover 20, the plurality of wiring patterns 24 and the cable connecting terminals 26 are formed on the bottom surface 68 of the recessed portion 67, the hole portions 68a, and the rear surface of the cable connecting portion 25 with the MID technology.

In the distal end cover 20, finally by reflow soldering, the respective solder bumps 45a and 46a are melted at the plated portions of the wiring patterns 24 formed respectively in the hole portions 68a, to electrically connect the wiring patterns 24. Note that, in the distal end cover 20, the respective hole portions 68a may be formed simultaneously with the activation of the bottom surface 68 of the recessed portion 67 by applying the laser light thereto.

Note that a filling agent is applied to the recessed portion 67 so as to cover the plurality of wiring patterns 24, to thereby ensure the insulation property and the water-proof property of the distal end cover 20.

As described above, the distal end cover 20 is configured such that the illumination module 45 as the illumination optical system and the camera module 46 as the image pickup optical system, which are electronic components, are insert-molded. Such a configuration of the distal end cover 20 can eliminate secondary processing for assembling the electronic components such as the illumination module 45 and the camera module 46, to thereby reduce the manufacturing cost and increase the durability by the electronic components and the resin being integrated.

Thus, the endoscope 1 of the present embodiment includes, at the distal end portion 8 of the insertion portion 5, the distal end cover 20 in which the electronic components are insert-molded, and the circuits are formed by using the MID technology. With such a configuration, similarly as in the first embodiment, the endoscope 1 is capable of achieving the ensuring of the mechanical strength and the durability simultaneously with the reduction of the manufacturing cost.

### First Modification Example

In the distal end cover 20, an optical system unit 30 including a resin frame 29 may be mounted, as shown in FIG. 27 and FIG. 28. In the resin frame 29, the parts around the illumination module 45 as the illumination optical system and the camera module 46 as the image pickup optical system are solidified by a resin.

The optical system unit 30 is mounted on the bottom surface 23 of the recessed portion 22 of the distal end cover 20, as shown in FIG. 29 and FIG. 30. The optical system unit 30 is configured such that the solder bumps 45a and 46a are soldered respectively to the illumination optical system terminals 24a and the image pickup optical system terminals 24b. Then, the filling agent 44 as a filler such as an underfill agent is applied to the recessed portion 22 to be molded so as to surround the periphery, of the optical system unit 30, and solidified by heat processing or the like.

Note that the optical system unit 30 is configured such that the illumination window 41 of the illumination module 45 and the observation window 42 of the camera module 46 are exposed on one surface side. Then, the optical system unit 30 is configured such that the resin frame 29 is formed so as to allow the respective solder bumps 45a and 46a to protrude on the other surface side.

### Second Modification Example

As shown in FIG. 31 and FIG. 32, the distal end cover 20 is configured to be divided into two parts. In the distal end cover 20, the circuits are formed by using the MID technology. The distal end cover 20 may be configured by bonding resin molded bodies, i.e., a first resin member 20a and a second resin member 20b by adhesion or the like. In the first resin member 20a, the electronic components such as the illumination module 45 and the camera module 46 are mounted. The second resin member 20b includes an opening portion 21 which is the raising base placing portion as a part in which the raising base 32 is arranged.

The distal end cover 20 has a configuration in which the plurality of wiring patterns 24 provided on the bottom surface 23 of the recessed portion 22 and the plurality of cable connecting terminals 26 provided on the plane of the cable connecting portion 25 are formed only in the first resin member 20a by using the MID technology. Therefore, in the distal end cover 20, the second resin member 20b can be made of a resin different from the one used for the first resin member 20a.

### Third Modification Example

The endoscope 1 may be configured such that, instead of the illumination module 45 as the illumination optical system mounted in the distal end cover 20, a distal end part of a light guide bundle 71 configured to guide illumination light is disposed in the distal end cover 20 by insert molding, as shown in FIG. 33.

In addition, as a configuration in which the light guide bundle 71 is not insert-molded in the distal end cover 20, an illumination optical system placing hole 72 may be formed in the distal end cover 20, as shown in FIG. 34.

The endoscope 1 in each of the above-described first embodiment and second embodiment, and each of the modification examples is configured as a single-use endoscope, to thereby be capable of achieving both the reduced cost and ensuring of the strength of the structure of the distal end portion 8. In other words, the structure of the distal end portion 8 of the endoscope 1 employs the hybrid structure of resin structure and metal structure.

In addition, in the side-view or oblique-view endoscope 1 including a raising base 32 for raising and lowering the treatment instrument or the like, the distal-most bending piece 50 as the base part of the raising base 32 which requires a mechanical strength is made of metal, and the distal end cover 20 for covering the raising base 32 is made of resin.

Then, the wiring patterns 24 are formed on the surface of the distal end cover 20 by using the MID technology, and at least the camera module 46 which is an electronic component connected to the wiring patterns 24 is laid out in the distal end cover 20. Note that the electronic component also includes the illumination module 45. Such a configuration of the endoscope 1 can achieve the ensuring of the strength and the durability simultaneously with the reduction of the manufacturing cost of the distal end portion 8 of the insertion portion 5.

The invention recited in each of the above-described embodiments and the modification examples is not limited to the one shown in each of the embodiments and the modification examples, and various modifications are possible at the practical stage in a range without departing from the gist of the invention. Furthermore, each of the above-described embodiments and modification examples includes the invention at various stages, and various inventions can be extracted by appropriately combining a plurality of disclosed constituent elements.

For example, even if some of the constituent elements are removed from all the constituent elements shown in the above embodiments and modification examples, a configuration from which the constituent elements are eliminated can be extracted as an invention insofar as the recited problem can be solved and the recited effects of the invention can be obtained.

What is claimed is:

1. A distal end portion of an endoscope comprising:
a moving mechanism configured to change a protruding direction of a treatment instrument;
a base made of metal and including a supporting portion configured to rotationally support the moving mechanism;
a cover member made of resin, the cover member being fitted to the base so as to cover the moving mechanism and including an opening portion formed at a position at which the moving mechanism is placed;
an electronic component disposed in the cover member; and
a wiring pattern electrically connected to the electronic component, the wiring pattern being provided in the cover member.

2. The distal end portion of the endoscope according to claim 1, wherein the electronic component is integrated, by insert molding, with a resin material that forms the cover member.

3. The distal end portion of the endoscope according to claim 1, wherein the cover member is a molded interconnect device in which the wiring pattern is formed.

4. The distal end portion of the endoscope according to claim 1, wherein the cover member is configured by two resin molded bodies being bonded to each other, the two resin molded bodies including a first resin member in which the wiring pattern is formed and the electronic component is mounted, and a second resin member in which the opening portion is formed.

5. An endoscope comprising:
an insertion body configured to be inserted into a subject;
a moving mechanism configured to change a protruding direction of a treatment instrument configured to be inserted through the insertion body;
a base made of metal and including a supporting portion configured to rotationally support the moving mechanism;
a cover member made of resin, the cover member being fitted to the base so as to cover the moving mechanism and including an opening portion formed at a position at which the moving mechanism is placed;
an electronic component disposed in the cover member; and
a wiring pattern electrically connected to the electronic component, the wiring pattern being provided in the cover member.
